(19) United States
(12) Patent Application Publication
(10) Pub. No.: US 2014/0193769 A1
(43) Pub. Date: Jul. 10, 2014

(54) Multi-Axial Positioning Dental Drill Guide Assembly

(71) Applicant: innoDent LLC, Colorado Springs, CO (US)

(72) Inventor: William J. Mackey, Colorado Springs, CO (US)

(73) Assignee: innoDent LLC, Colorado Springs, CO (US)

(21) Appl. No.: 14/150,499

(22) Filed: Jan. 8, 2014

Related U.S. Application Data
(60) Provisional application No. 61/750,720, filed on Jan. 9, 2013.

(51) Int. Cl. A61C 1/08; A61C 8/00; A61K 6/10; A61B 6/00; A61B 6/02; A61C 9/00; A61C 3/02; A61B 6/14

(52) U.S. Cl. CPC A61C 1/084; A61C 1/085; A61C 3/02; A61C 8/0089; A61B 6/14; A61B 6/4085; A61B 6/02; A61C 9/0006; A61K 6/10
USPC 433/29; 433/75; 433/76; 433/37; 433/48

(57) ABSTRACT

A dental device is provided for guiding a drill bit. The dental device comprises a platform adapted to be mounted on a person's jaw. At least one drill guide is coupled to the platform and adapted for translational and rotational adjustment with respect to the platform. In one embodiment, the platform comprises a single bite registration receiver. In another embodiment, the platform comprises a triangular frame, including a cross-arch receiver provided for stability. A third embodiment provides a platform for guiding four drill bits. In each embodiment, a multi-axis positioning assembly enables setting and adjustment of the entry point and angular orientation of each drill guide and provides a surgeon with ample room to view the procedure as it is performed.

### RELATED APPLICATIONS

This application is a non-provisional of, and claims the benefit of, my U.S. Patent Application No. 61/750,720, filed Jan. 9, 2013, and entitled “Dental Osteotomy Appliance and Preparation Method,” which is herein incorporated by reference and hereinafter referred to as the “Provisional Application.”

This application is also related to the following concurrently-filed applications, both of which are incorporated herein by reference:

| Docket | | Serial |
|---|---|---|
| No. | Title | No. |
| 2040.002 | Dental Drill Platform with Adjustable Drill Bushing | tbd |
| 2040.003 | Dental Osteotomy Appliance and Preparation Method | tbd |

### FIELD OF INVENTION

This invention relates generally to restorative dentistry, and more particularly, to surgical drill guides for dental implants.

### BACKGROUND

For centuries, the standard treatment for tooth loss was full or partial removable dentures to replace some or all of a person's teeth. Dentures have numerous disadvantages. Ill fitting dentures interfere with eating, speaking, and smiling. Years of denture use can wear away the basal and alveolar bone of the upper and lower jaws, causing the chin to jut forward, shortening the distance between the chin and the nose, and deepening the groove between the nose and the corner of the lip.

Modern medicine introduced endosseous dental implants as an alternative to traditional dentures. Dental implants are typically rods or posts that are inserted into the jawbone. They appear similar to an actual tooth root (i.e., they have “root-form”), except that they are typically threaded. The implants, usually formed of titanium, a titanium alloy, or zirconium dioxide, are placed in approximately the same place where a missing tooth's root once existed. The implant surfaces are typically mechanically and/or chemically treated or coated to promote osseointegration.

Significant care and preparation are required in advance of the dental osteotomy. An implant needs to be spaced away from nearby tooth roots to avoid damaging them. Care must also be taken to prevent either the drill or the implant from contacting or penetrating the inferior alveolar nerve or the sinus. An adequate amount of jawbone is needed to securely anchor the implant. A panoramic X-ray or a computer assisted tomography scan is commonly used to help identify whether the maxillary or mandibular alveolar bone has sufficient height, depth, and width to support the implant. If the amount of available alveolar bone is insufficient, bone may first be grafted into the area.

It is important to know the shape and dimensions of the jawbone prior to surgery, and to guide and orient the drill properly during surgery. Accordingly, it is conventional practice to prepare a customized “surgical stent” or template for guiding the dental drill. Typically, a mold is made from the patient's teeth impressions. Then, a study model or dental cast is made of the patient's jaw from the molds. Third, a retainer-like surgical stent or template, contoured and customized to fit the teeth and palatal and ridge contours of the patient's jaw, is made from the model. A guide is milled and/or mounted in the dental stent and positioned over the appropriate edentulous jaw site for positioning and axially aligning the drill bit.

At the time of surgery, the surgical area is anesthetized, a flap incision is made in the gum to expose the underlying bone, and the stent is placed over the jaw. A pilot hole is drilled into the jaw and then widened using a series of progressively wider drill bits. The implant is placed in the hole, with its top positioned flush with or just above the crest of the bone. A temporary cover cap is screwed into the implant and the gum stitched back over the implant. Several months later, after there has been sufficient time for osseointegration, the cover cap is removed and replaced with a healing cap that sticks out above the gum. After several more months for the gum to form around the healing cap, the healing cap is replaced with a post that supports the dental prosthesis (i.e., typically a crown, an implant-supported bridge, or denture).

Many variations to the processes and implements described above are known in the art.

Because an aspect of the present invention relates to an improved dental stent (alternatively referred to as a “stint” and sometimes a “splint”), further discussion of conventional methods for customizing, calibrating, and placing dental stents is merited.

U.S. Pat. No. 5,613,852 to Bavitz discloses a full-jaw acrylic dental stent in which guide sleeves are inserted to orient a drill. Bavitz also discloses a method of preparing and using the guide stent. An impression is taken of the patient's teeth in the vicinity of a void where the dental implants will be implanted. A model of the teeth is cast from the impression. After mounting the model to a drill press, a pilot hole is drilled into the void in the model. A guide rod is threaded into the pilot hole. Dental acrylic is used to fabricate a tooth-conforming jaw stent. The guide rod is removed and replaced with guide sleeves having progressively larger internal diameters.

U.S. Pat. No. 5,636,986 to Pezeshkian discloses drill guides configured in the shape of two or more adjacent teeth. Each drill guide carries drill bushings for guiding a drill bit. After a first hole is drilled into the jawbone, a pin is inserted into the drill bushing and hole. The drill guide is then pivoted about the pin axis to an appropriate position to enable the surgeon to drill an adjacent second hole for an adjacent implant.

U.S. Pat. No. 5,718,579 to Kennedy discloses a kit and method for creating a customized dental stent. Before forming the stent, a model is made of the person's dentition and edentulous area and desired drill hole point marked on the model. A cementitious composition is molded to the contours of the model to form the stent. The stent includes a tab that covers the edentulous region. The tab is marked and a hole drilled at the marked location. A sleeve is then placed in the hole, manipulated via a dowel into the desired axial alignment, and additional cementitious material applied to cement the sleeve in place.

U.S. Pat. No. 5,967,777 to Klein et al. describes several prior art approaches to dental osteotomy. In the first approach, a patient wears a “CT scan appliance” as a computerized tomography scan is made of the patient's jawbone. Software transforms the CT scan data into virtual 2D models that simulate a dental implant. Then the CT scan appliance is modified into a surgical template. During surgery, the surgeon conventionally “eyeballs” the trajectory and angular orientation of the dental implant drill through the hole in the surgical template. In the second approach, CT scan data is used to fabricate a clear plastic acrylic model of a patient's jaw. A hole is drilled into the model and a replica of an implant inserted into the hole. A cylinder is attached to the top of the replica and more acrylic added to encase the cylinder.

In Klein's preferred approach, a plastic replica of teeth to be implanted in a gap is created with anchors on either side for attaching the replica to still-existing teeth on either side of the gap. Fiducial markers are attached to the plastic replica with acrylic or other suitable adhesive. After placing the plastic replica in the patient's mouth, a CT scan is performed. Surgical simulation software is used to determine an optimal position and angulation for the dental implants. These coordinates are then transferred to a computer-driven milling machine to drill a hole into the plastic replica, converting the plastic replica into a surgical template. The fiducial markers are removed and a master cylinder is inserted into the hole. The master cylinder provides an inner axial bore for receiving removable first and second drill bushings and an implant bushing. A set screw locks the bushings to the master cylinder.

U.S. Pat. No. 6,971,877 to Harter describes a full-mouth dental stent having a ball-and-socket joint between a bushing holder that snap-fits into the dental stent and a drill bushing. Once the drill bushing is angularly oriented to the desired position, a bonding material is applied to hold the bushing stationary.

These and other prior systems have several impediments. Known drill stents do not enable fine-tuned, multi-axial translation adjustments after the drill guide has been attached to the drill guide support base. Once a hole is drilled in the mold to receive a cylindrical sleeve or bushing holder, the position of the cylindrical sleeve or bushing holder is fixed and cannot be readjusted. Typically, the orientation of the drill bushing is also fixed, or if not—as in the case of Harter's ball-and-socket joint—changing the orientation of the drill bushing necessarily moves the drill bit entry point. Also, known drill stents that make use of molds that conform to the contours of the patient's dentition also encapsulate and enclose the drill area, occluding or blocking the surgeon's ability to view the drill area as the osteotomy is being performed and interfering with irrigation and evacuation.

### SUMMARY

Characterized one way, the invention comprises a dental device for guiding a drill bit, the dental device comprising a platform adapted to be mounted on a person's jaw and at least one drill guide coupled to the platform and adapted for translational and rotational adjustment with respect to the platform.

Characterized a second way, the invention comprises a dental device for guiding a drill bit, the dental device comprising a platform adapted to be mounted on a person's jaw and at least one carrier coupling a drill bushing to the platform.

Characterized a third way, the invention comprise a dental device for guiding a drill bit, the dental device comprising one or more locking receivers, each locking receiver being adapted to receive an application of impression material and, after the impression material is conformed to a patient's dentition, retain the impression material to the receiver, and one or more drill guides coupled to the locking receivers.

Characterized a fourth way, the invention comprises a dental device for guiding a drill bit, the dental device comprising a platform adapted to be mounted on a person's jaw, an adjustable drill guide coupled to the platform, and a procedure viewing window formed in the platform around the drill guide. The procedure viewing window is formed by a viewable clearance between the drill guide and an edentulous region of the person's jaw and facilitates flushing and evacuation of a patient's mouth during the dental procedure.

Characterized a fifth way, the invention comprises a method of preparing for a dental osteotomy. First, impression material is applied to a dental appliance. Then, the impression material is conformed to a person's dentition or a model of a person's dentition. After the impression material cures, a drill path alignment tool is inserted through a drill guide coupled to the dental appliance. The drill path alignment tool is then moved to a selected position and angular orientation, relative to the dental appliance, that is consistent with a position and angular orientation of a planned dental implant. Movement of the drill path alignment tool moves the drill guide to the selected position and angular orientation and configures the dental appliance and associated drill guide to guide a dental osteotomy.

There are also various aspects that further characterize some embodiments of the invention. In one aspect, the dental device provides at least a three-axis positioning assembly for the at least one drill guide. Stated differently, an adjustable drill guide positioning assembly couples the at least one drill guide to the platform.

In another aspect, the at least one drill guide is operable to receive a drill path alignment tool and, while the drill path alignment tool's end is maintained in contact with a desired drill entry point, be adjusted by the drill path alignment tool into a desired angular orientation and lateral position with respect to the platform.

In another aspect, one or more mechanical locks are provided to lock the at least one drill guide into the desired angular orientation and lateral position after the alignment tool has been used to set the desired angular orientation and lateral position.

In another aspect, the dental device is operable to be placed on the person's jaw, imaged with respect to the person's jaw, removed from the person's jaw, and the mechanical locks are operable to be unlocked and re-locked to further adjust the at least one drill guide to a second, image-calibrated angular orientation and lateral position.

In one embodiment, the platform comprises a plurality of receivers, each receiver being adapted to fit over a tooth portion or ridge portion of the person's jaw. Moreover, the plurality of receivers are adapted to be distributed across opposite sides of a dental midline of a person's jaw to stabilize the platform. In particular, first and second of the plurality of receivers are adapted to fit on distal and mesial sides of an edentulous area of the jaw, and a third of the plurality of receivers is a cross-arch receiver adapted to fit over a third tooth portion or ridge portion of the person's jaw. The third receiver is adjustably moveable relative to the first and second receivers. Also, an angle between a line connecting the first and second receivers and a line connecting the first and third receivers is adjustable between narrow and wide angular limits to fit a variable range of mouth sizes and placements of the platform to a person's jaw. Furthermore, at least the first and second receivers are linked by a mechanically deformable bar, wherein deformation of the bar translates the drill guide along a coronal or apical direction relative to the person's jaw.

In another embodiment, the dental device has a plurality of receivers that are distributed on distal and mesial sides of multiple edentulous areas of the jaw. Also, at least two drill guides are coupled to the platform and adapted for and rotational adjustment with respect to the platform, with one drill guide oriented over a first edentulous area of the jaw, and another drill guide is oriented over a second edentulous area of the jaw. In one particular version, at least four drill guides are coupled to the platform and adapted for and rotational adjustment with respect to the platform, with two drill guides coupled to the platform between a first pair of the plurality of receivers, and two more drill guides coupled to the platform between a second pair of the plurality of receivers.

Generally, although not necessarily, each receiver comprises a pad with one or more indentions, such as putty receptacles, formed in the pad for retaining impression material such as dental putty. Each putty receptacle is formed in the pad with an undercut to retain the putty to the pad.

As noted earlier, the drill guide, in at least one embodiment, comprises a carrier and a drill bushing. Each carrier is operable to translate the corresponding drill bushing to a selected position between both buccal and lingual limits and distal and mesial limits. This is made possible by a slot disposed in each carrier, the slot facilitating pivotal and linear translational movement of the carrier with respect to the platform. A mechanical lock enables the carrier to be locked into a fixed position relative to the platform. The drill bushing is pivotally coupled along at least one axis, and in one alternative two axes, to the carrier. Also, the drill bushing is configured with a bore and a seat adapted to receive a flanged pilot bushing for drilling of a pilot hole into a jawbone.

Also in two embodiments, the drill bushing is borne by a drill bushing housing that is pivotally coupled to the carrier. A locking screw is disposed through the carrier that enables the drill bushing housing to be locked into a selected angular orientation. In one embodiment, the drill bushing housing comprises a yoke joined to a cylinder formed for rotation within a pivot bore at the end of the carrier. The pivotal coupling between the drill bushing housing and the carrier enables a distal-mesial angular orientation of the drill bushing to be set. Moreover, a drill bushing is pivotally mounted within the yoke of the housing to enable a buccal-lingual angular orientation of the drill bushing to be set. In the other embodiment, the drill bushing housing does not include a yoke, but rather comprises a bushing seat coupled to a cylinder formed for rotation within a pivot bore at the end of the carrier. The pivotal coupling between the drill bushing housing and the carrier enables a buccal-lingual angular orientation of the drill bushing to be set.

In another aspect, the dental device provides structure for locking movable parts with chemical adhesive. In one embodiment, a basin is disposed in the top surface of each carrier for receiving and containing the adhesive and funneling some of the adhesive toward the port. A port is provided the carrier to convey chemical adhesive from the basin to a bottom surface of the carrier. The platform to which the adhesive flows is directed into a plurality of canals that distribute the adhesive between the flat top surface of the platform and the flat bottom surface of the carrier.

The invention described herein is broad enough to encompass embodiments that do not appropriate all, some, or any of these cited advantages. Indeed, different embodiments described herein provide different subsets of the aforementioned advantages. Accordingly, the invention disclosed herein encompasses dental devices that provide fewer or more degrees of freedom, provide different numbers of drill guides, or use differently-configured platforms, frames, carriers, and drill guides. The scope of any given claim will be set forth by the claim language itself.

These and other objects, features, and advantages of the present invention will be readily apparent to those skilled in the art from the following detailed description taken in conjunction with the annexed sheets of drawings, which illustrate the invention.

### BRIEF DESCRIPTION OF THE DRAWINGS

FIG. 1 is a top perspective view of a right-sided embodiment of a dental device for guiding a drill bit, mounted on a model of a person's dentition.

FIG. 2 is top perspective view of a left-sided embodiment of a dental device for guiding a drill bit, mounted over a different edentulous portion of a model of a person's dentition.

FIG. 3 is a top perspective view of the dental device of FIG. 1, with the dentition model removed.

FIG. 4 is a bottom perspective view of the dental device of FIG. 2, also with the dentition model removed.

FIG. 5 is a front perspective view of another embodiment of a dental device for guiding a drill bit, mounted on a model of a person's dentition.

FIG. 6 is another front perspective view of the dental device of FIG. 5, shown mounted over a different edentulous portion of a model of a person's dentition.

FIG. 7 is a graph illustrating the lateral and rotational adjustability of the drill guide.

FIG. 8 is a rear perspective view of the dental device of FIG. 5 that illustrates different aspects of the dental device.

FIG. 9 is another rear perspective view of the dental device of FIG. 5.

FIG. 10 is a top perspective view of the dental device of FIG. 5.

FIG. 11 is a side perspective view of the dental device of FIG. 5 that illustrates a drill path alignment tool positioned in the drill guide.

FIG. 12 is a top view of an unmounted dental device.

FIG. 13 is a bottom perspective view of the dental device of FIG. 12 that illustrates one embodiment of the bite registration receivers incorporated into the dental device.

FIG. 14 is another bottom perspective view of the dental device of FIG. 12 that illustrates a different perspective of the drill guide and two additional bushings used to receive rods that adjustably couple a main platform piece to a cross-arch stabilizer.

FIG. 15 is a top perspective view of the dental device of FIG. 12, with the drill guide and carrier removed, that illustrates the telescoping action and pivoting action of respective ones of the rods.

FIG. 16 is a top perspective view of the main platform piece, with the drill guide and carrier removed, that illustrates an adhesive distribution network to distribute a chemical adhesive between the arm and the platform in order to lock the arm into a fixed position relative to the platform.

FIG. 17 is a bottom perspective view of the main platform piece, illustrating how it comprises two bite registration receivers bridged by a mechanically deformable elevator bar.

FIG. 18 is a perspective view of a top side of the cross-arch stabilizer.

FIG. 19 is a perspective view of a bottom side of the cross-arch stabilizer.

FIG. 20 depicts the slide rod that enables adjustment of the spacing between the smaller of the main platform piece's two receivers and the cross-arch stabilizer.

FIG. 21 depicts the pivot rod that enables adjustment of the spacing between the larger of the main platform piece's two receivers and the cross-arch stabilizer.

FIG. 22 illustrates a front view of one embodiment of an adjustable carrier for carrying the drill guide and setting the entry point of a drill bit.

FIG. 23 illustrates a back view of the adjustable carrier of FIG. 22, with the multi-axially adjustable drill guide removed.

FIG. 24 illustrates a front view of the adjustable carrier of FIG. 22, also showing the drill guide removed.

FIG. 25 is a front perspective view that illustrates one embodiment of a drill guide that enables adjustment of the pitch and roll angles of a drill bit.

FIG. 26 is a rear perspective view of the drill guide of FIG. 25.

FIG. 27 illustrates a yoke that rotates to provide mesial-distal rotation of a drill bit.

FIG. 28 illustrates a bushing that pivots to provide buccal-lingual rotation of a drill bit.

FIG. 29 illustrates one embodiment of a pilot bushing configured to sit in the bushing of FIG. 28.

FIG. 30 is a top perspective view of another embodiment of a dental device for guiding a drill bit, mounted on a model of a person's dentition.

FIG. 31 is an elevated rear perspective view of the dental device of FIG. 30.

FIG. 32 is a top perspective view of the dental device of FIG. 30, with the dentition model removed.

FIG. 33 is a bottom perspective view of the dental device of FIG. 30, with the dentition model removed.

FIG. 34 is a bottom perspective view of the frame of the dental device of FIG. 30, with the dental guides removed.

FIG. 35 is a top perspective view of the frame shown in FIG. 34.

FIG. 36 is a top rear perspective view of a left-sided carrier of the dental device of FIG. 30.

FIG. 36 is a top front perspective view of a right-sided carrier of the dental device of FIG. 30.

FIG. 37 is a bottom rear perspective view of a left-sided carrier of the dental device of FIG. 30.

FIG. 38 is a bottom front perspective view of a right-sided carrier of the dental device of FIG. 30.

FIG. 40 is a perspective view of a left-sided bushing for the dental device of FIG. 30.

FIG. 41 is a perspective view of a right-sided bushing for the dental device of FIG. 30.

FIG. 42 is a bottom perspective view of another embodiment of a left-sided drill guide platform or frame.

FIG. 43 is another bottom perspective view of another embodiment of a left-sided drill guide platform or frame.

FIG. 44 is a perspective view of a right-sided drill guide platform or frame mounted on a model of a person's dentition.

FIG. 45 is another perspective view of a right-sided drill guide platform or frame mounted on a model of a person's dentition.

### DETAILED DESCRIPTION

Before the subject invention is described further, it is to be understood that the invention is not limited to the particular embodiments of the invention described below or depicted in the drawings. Many modifications may be made to adapt or modify a depicted embodiment without departing from the objective, spirit and scope of the present invention Therefore, it should be understood that, unless otherwise specified, this invention is not to be limited to the specific details shown and described herein, and all such modifications are intended to be within the scope of the claims made herein.

It is also to be understood that terms of art and words in general carry a range of meanings. Language is an imprecise medium of communication. The terminology and grammar employed in this specification is for the purpose of describing and explicating particular embodiments. Unless the context clearly demonstrates otherwise, the particular terms and grammatical structure employed should be liberally construed.

The accompanying drawings depict a dental device for guiding a drill bit into an edentulous area of an alveolar bone. The dental device may be referred to by a number of alternative names, including but not limited to a dental appliance or dental stent.

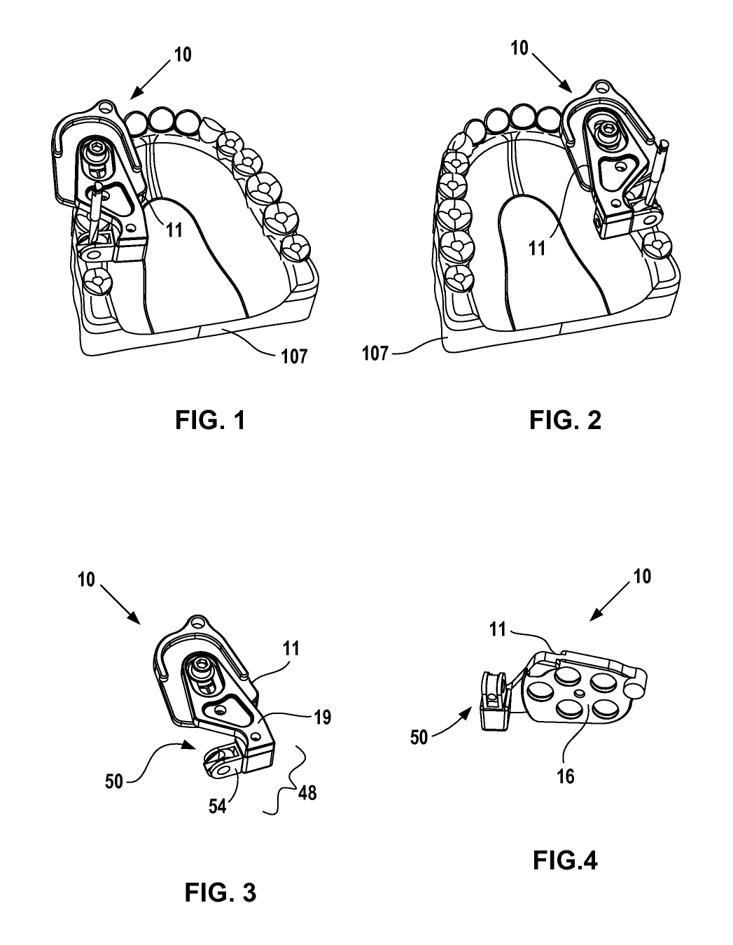

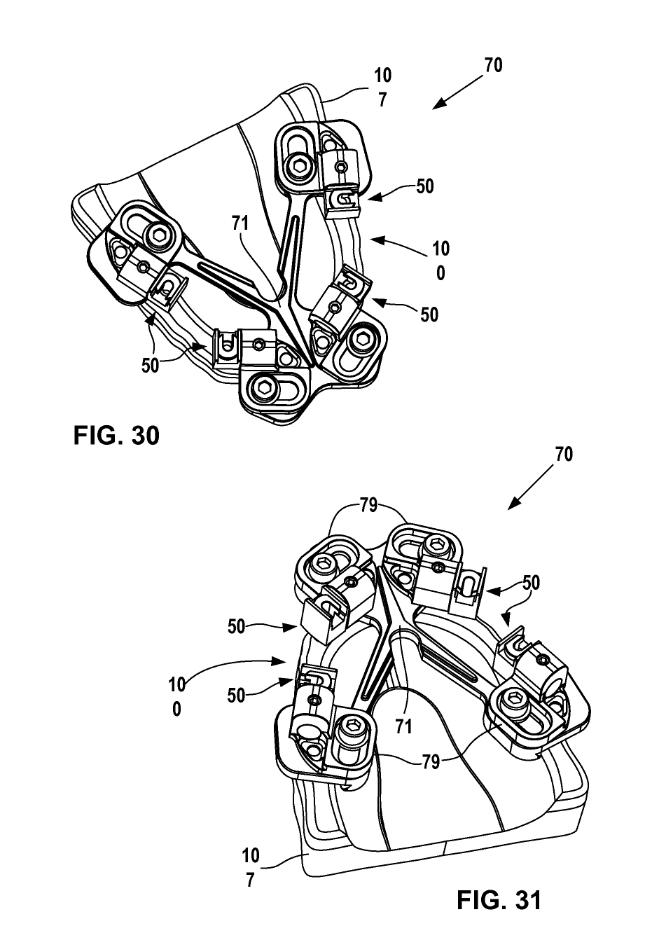

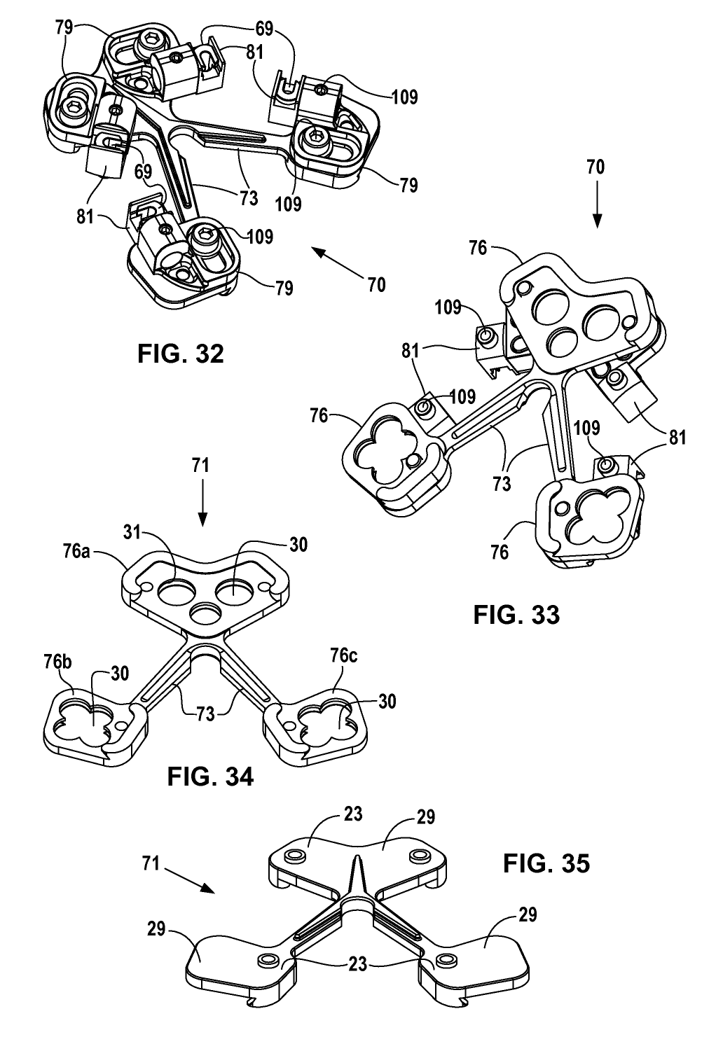

FIGS. 1-4 illustrate one embodiment of a dental device or appliance 10 for guiding a drill bit for use in dental surgery. The dental device 10 is shown mounted on a model 107 of a person's dentition. The dental device 10 comprises an adjustable drill guide 50 coupled to a frame, such as platform 11. Other platform configurations, such as those illustrated in 5-6 and FIGS. 30-35, are possible. The platform 11 mounts on a person's jaw and, because it incorporates a bite registration receiver 16, is configured to provide a platform that can stay stably seated on the person's jaw during surgery.

There are mirror-image opposite layouts of the dental device 10. The dental device 10 of FIGS. 1 and 3 are designed for operations on the right mandible and left maxilla. The dental device 10 of FIGS. 2 and 4 are designed for operations on the left mandible and right maxilla.

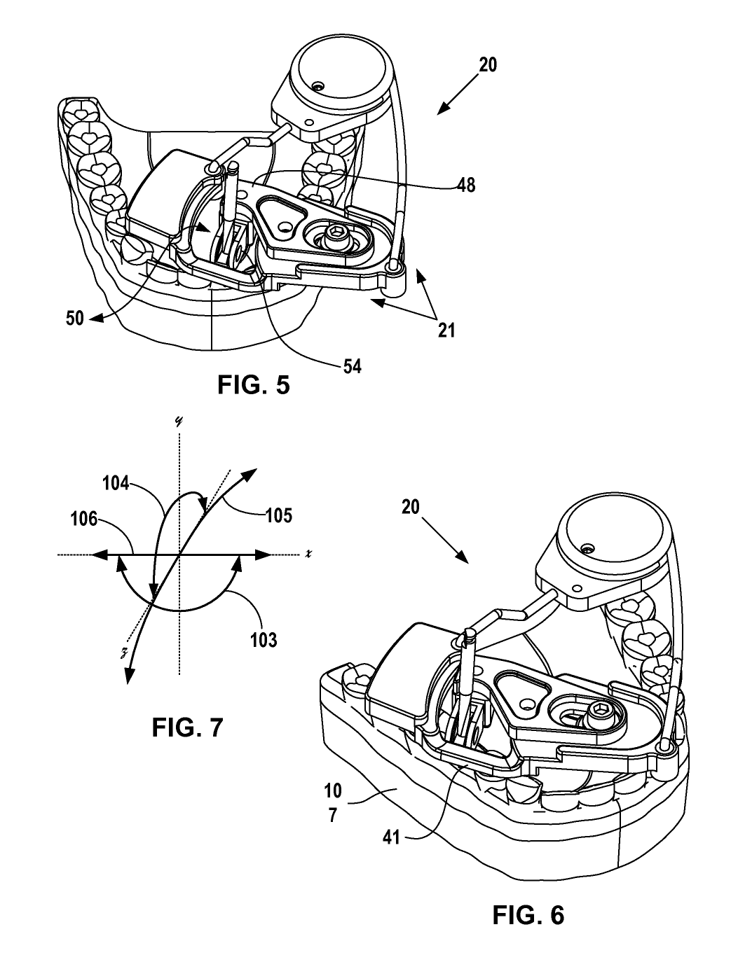

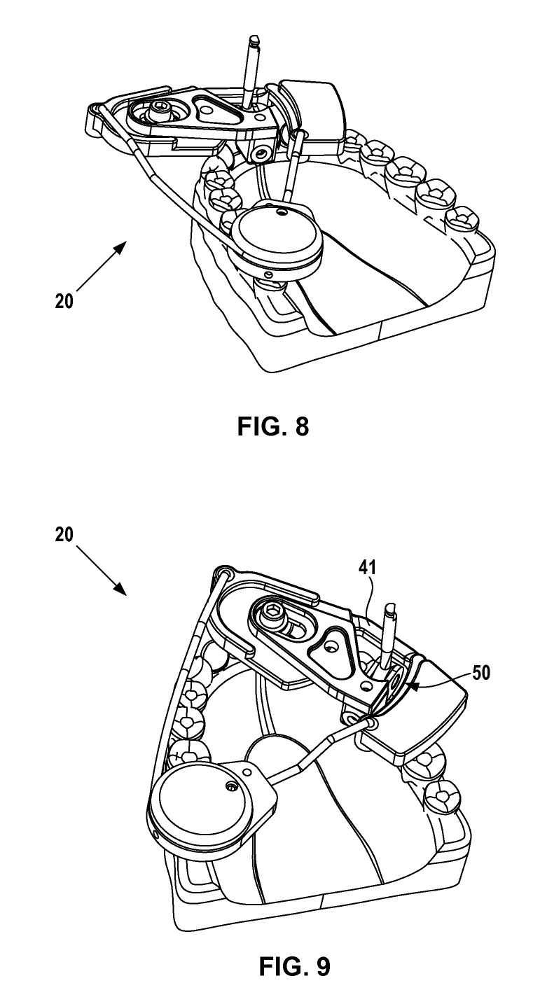

As just noted, a drill guide 50 is coupled to the platform 11 and is adjustably positionable with respect to the platform 11. In the present embodiment, the drill guide 50 comprises a drill bushing housing or assembly 54. A multi-axis drill guide positioning assembly 48 carries the drill bushing housing 54 for four-axis translational and rotational adjustment with respect to the platform 11. Because the dental device 10 of FIGS. 1-4 shares much in common with the dental device of FIGS. 5-29, this description now turns to describing the second embodiment before discussing the shared details of both.

FIGS. 5 and 6 illustrate a second embodiment of a dental device or appliance 20 for guiding a drill bit for use in dental surgery. The dental device 20 is shown mounted on a model 107 of a person's dentition. The dental device 20 comprises an adjustable drill guide 50 coupled to a frame, such as tripod platform 21. The platform 21 mounts on a person's jaw and is configured to provide a platform that can stay stably seated on the person's jaw during surgery.

Like the embodiment shown in FIGS. 1-4, a drill guide 50 is coupled to the platform 21 and is adjustably positionable with respect to the platform 21. Similarly, the drill guide 50 comprises a drill bushing housing or assembly 54. A multi-axis drill guide positioning assembly 48 carries the drill bushing housing 54 for four-axis translational and rotational adjustment with respect to the platform 21. These four degrees of freedom are exemplified in FIG. 7, discussed further below.

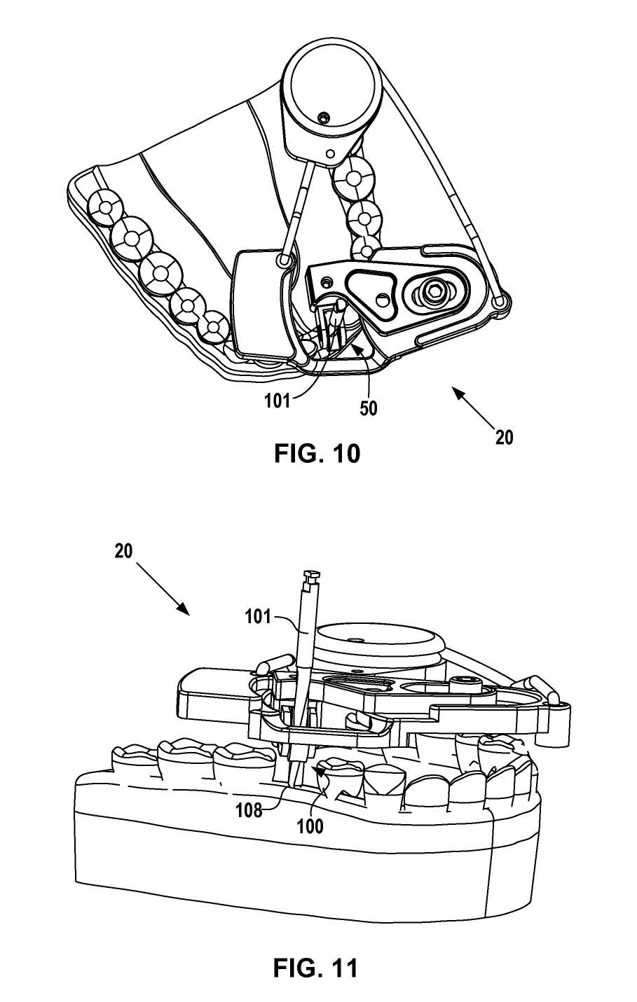

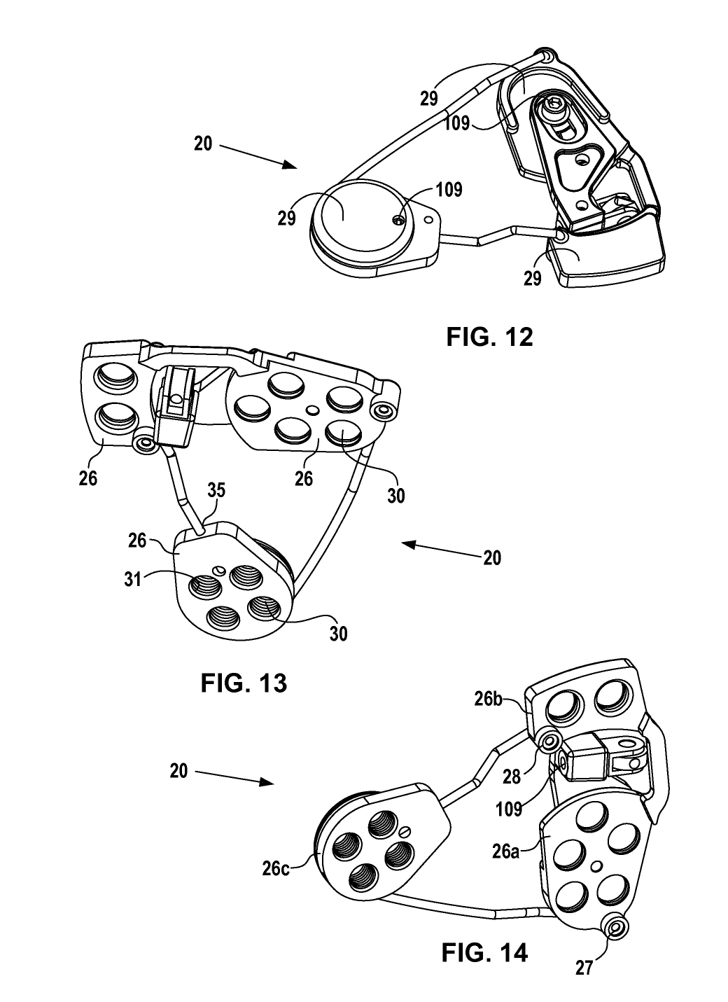

As best illustrated in FIGS. 10 and 11, the drill guide 50 is operable to receive a drill path alignment tool 101 such as a rod that includes an end tip 108. While the drill path alignment tool 101's end tip 108 is maintained in contact with a desired drill entry point, the drill path alignment tool 101 is adjusted into a desired angular orientation (both roll and pitch angles) and lateral position with respect to the platform 21, translating and angularly positioning the drill guide 50 with it. Mechanical locks 109, such as locking screws (see, e.g., FIGS. 12 and 14), are then adjusted or tightened to lock the drill guide 50 into the desired angular orientation and lateral position. Significantly, the mechanical locks 109 can be unlocked and re-locked to enable the drill guide 50 to be fine-tuned to a second, more precisely calibrated angular orientation and lateral position.

Turning to FIGS. 12-21, the platform 21 comprises a plurality of bite registration receivers 26a-c (collectively 26), each receiver 26 being adapted to fit over a tooth portion (e.g., one or more teeth) or ridge portion of the person's jaw. Each receiver 26 receives and retains impression material, such as bite or dental putty, forming an anchor for the platform 21. More particularly, each receiver 26 comprises a pad 29 with a plurality of putty receptacles or indentions 30 formed in the underside of the pad 29 for receiving an application of and retaining an impression material. In one embodiment each putty receptacle 30 is formed with an undercut 31 to retain the impression material to the pad 29. After the impression material is conformed to a patient's dentition, the receptacles 30 retain the impression material to the receiver 26.

In the embodiment of FIGS. 12-21, the platform 21 comprises three triangularly-arranged receivers 26, the first (26a) and second (26b) of which are adapted to fit on teeth or ridge portions on opposite sides of an edentulous area of the jaw, and a third (26c) of which is adapted to fit over a third tooth portion or ridge portion of the person's jaw. The receivers 26 are approximately coplanar with each other.

Characterized another way, the platform 21 comprises a drill-guide-supporting base or main platform piece 22 (FIG. 16). At least a first one (26a), and preferably also a second one (26b), of the plurality of receivers 26 are disposed in the main platform piece 22 (FIG. 17). Another of the plurality of receivers 26 is a cross-arch stabilizer 26c (FIGS. 18, 19) configured to be anchored to a tooth portion (which may include multiple teeth) or gum ridge portion on an opposite side of the jaw (left or right), opposite the side of the jaw (right or left) on which at least a portion of the base is located. In this way, receivers 26 are disposed on both sides of the dental midline, stabilizing the platform 21. Furthermore, the cross-arch stabilizer 26c is moveable and lockable relative to the main platform piece 22, enabling the platform 21 to be adapted to different edentulous regions and different size mouths. In the present embodiment wherein the main platform piece 22 comprises two receivers 26, a mechanically deformable elevation bar 41 links the two receivers 26. Deformation of the elevation bar 41 into an arc, as exemplified in Photo 12 of the Provisional Application, translates the drill guide 50 in a vertical dimension (coronal or apical direction), enabling adjustment of the clearance between the drill guide 50 and the surgical entry point. The buccal position of the bar 41, relative to the two anchors it links, also forms a procedure viewing window 100 (FIG. 11) in the base. In one embodiment, the main platform piece 22 is a metal part, and the cross-arch stabilizer 26c is a plastic part.

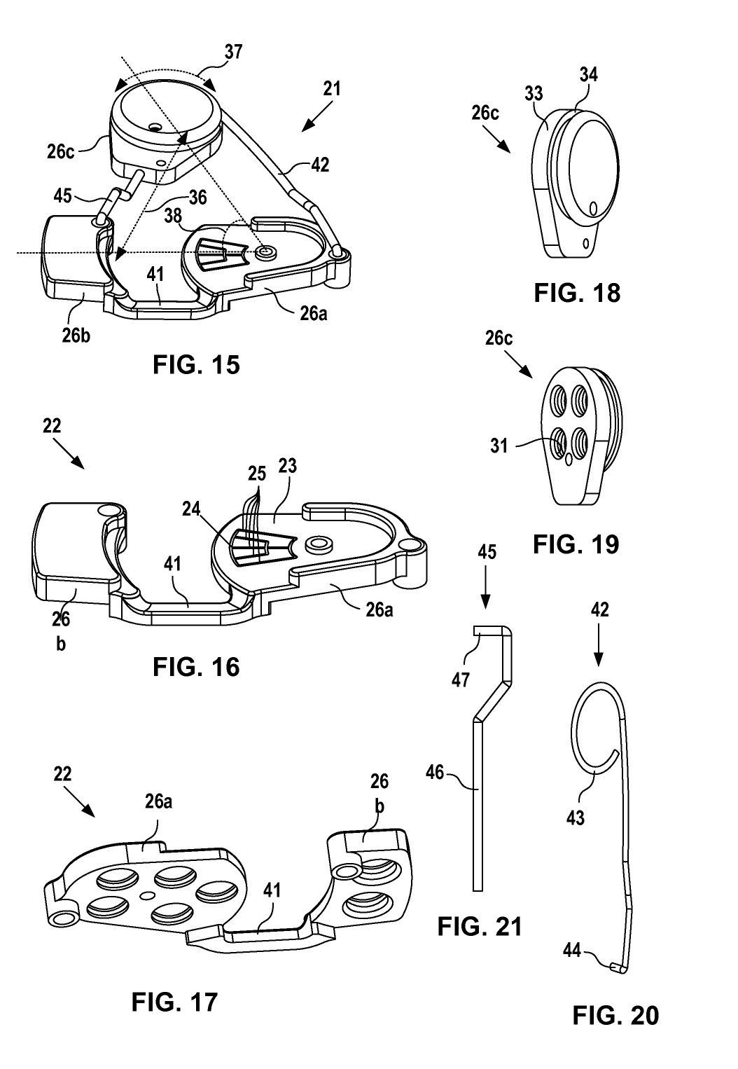

The cross-arch receiver 26c is moveable relative to the first and second receivers 26a and 26b. A pair of prismatic and revolute joints constrain the third receiver 26c for movement along a radial path 37 with respect to the first receiver 26a and along an axial path 36 with respect to the second receiver 26b (FIG. 15). In particular, the first and third receivers 26a and 26c are linked by a pivot bar (or rod) 42, and the second and third receivers are linked by a slide bar (or rod) 45. A curved slot 34 formed in an outer cylindrical portion 33 of the third receiver 26c carries a round spring end 43 of the pivot bar 42 (FIG. 20), guiding the third receiver 26c for pivotal radial movement with respect to the first receiver 26a. An axial diametrical slot 35 (FIG. 13) formed through the third receiver 26c carries an elongated linear end 46 of the slide bar 45 (FIG. 21). The pivot bar 42 and slide bar 45 each have 90-degree bends to provide short pivot ends 44, 47 opposite their cross-arch receiver engaging ends 43, 46, the pivot ends 44, 47 being disposed for pivotal movement within respective bushings 27 and 28 disposed in the first and second receivers 26a, 26b (FIG. 14).

An angle 38 between a line connecting the first and second receivers 26a, 26b and a line connecting the first and third receivers 26a, 26c is adjustable between wide and narrow angular limits and to fit a variable range of mouth sizes and to facilitate placement of the platform 21 over any edentulous region of a person's jaw. A mechanical lock 109, such as set screw or locking screw, is provided to lock the third receiver 26c into a fixed position relative to the first and second receivers 26a, 26b (FIG. 12).

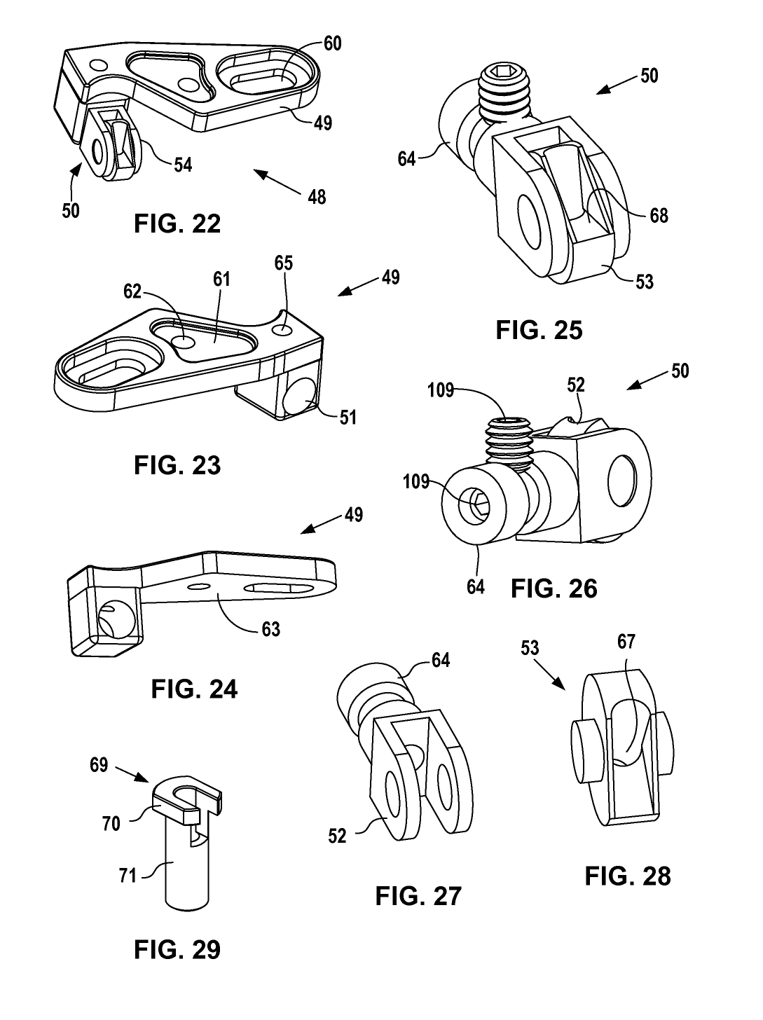

Turning to FIGS. 22-28, attention is now directed to aspects of the multi-axis drill guide positioning assembly 48, which is common to both the first embodiment (FIGS. 1-4) and the second embodiment (FIGS. 5-21). The drill guide positioning assembly 48 comprises a carrier 49 that couples the drill guide 50 (which here takes the form of a drill bushing housing or assembly 54) to the platform 11 or 21. In one embodiment, the carrier 49 comprises a member, such as an arm, that is pivotally and slidably joined, via a pin-in-slot joint, to the first receiver 26a. The carrier 49 carries the drill bushing housing or assembly 54 and translates it to a selected position between buccal and lingual limits 105 and between distal and mesial limits 106 (FIG. 7) across an approximately transverse plane. A slot 60 disposed in the carrier 49 (FIG. 22) and a substantially planar interface between the carrier 49 and the platform 21 facilitates selective translational movement of the carrier 49 via a pin-in-slot joint along a transverse plane. A mechanical lock 109, such as a locking screw and washer (FIG. 12), enable the carrier 49 to be locked into a fixed position relative to the platform 21.

Substantially planar facing portions 63 and 23 of the carrier 49 (FIG. 24) and platform 21 (FIG. 16) provide mating regions for applying adhesive to chemically lock the carrier 49 into a fixed position relative to the platform 21. An adhesive distribution system 24, such as a network of canals 25 positioned between a flat surface portion 23 of the platform 21 and a contacting surface portion 63 of the carrier 49, distribute the chemical adhesive between the carrier 49 and the platform 21 in order to lock the carrier 49 into a fixed position relative to the platform 21. A port 62 (FIG. 23) is also disposed in the carrier 49 to convey chemical adhesive from a top surface of the carrier 49 to a bottom surface of the carrier 49. A basin 61 is also disposed in the top surface of the carrier 49 for receiving and containing the adhesive and funneling some of the adhesive toward the port 62. The canal system 24 underneath the reservoir 61 and port 62 completes the distribution for an even displacement of adhesive between the carrier 49 and the main platform piece 22. Alternatively, the port 62 may be used to receive a secondary locking screw (not shown) to securely lock the carrier 49 in place without using adhesive.

In an alternative embodiment, not shown, the drill guide 50 takes the form of a ball and socket joint. But as noted above, the drill guide 50 in this embodiment takes the form of a bushing housing 54. The drill guide 50 is affixed to the forward or buccal end of the carrier 49, so that the carrier 49 is disposed to the rear of the drill guide 50 it carries. In the present embodiment—which facilitates a wider range of angular drill guide orientations and can fit into edentulous gaps as small as 5 mm (or smaller) while still guiding drill bits as wide as 4 mm—the drill guide 50 comprises a dual axle joint, such as a yoke 52 and parent drill bushing 53.

The yoke 52 is pivotally coupled to the carrier 49 along an approximately coronal axis for distal-mesial angular orientation of the yoke and the parent bushing 53 it carries. The yoke 52 is mounted to a cylindrically-shaped pivot 64 that is borne by a carrier bore 51. A locking screw 109 (FIG. 26) disposed orthogonally of the carrier bore 51, through a vertical hole 65 (FIG. 23) disposed in the carrier 49, enables a surgeon or technician to lock the yoke 52 into a selected distal-mesial orientation (or roll angle) 103 (FIG. 7).

The parent bushing 53 is pivotally mounted within the yoke 52 along an approximately sagittal axis for selectable buccal-lingual angular orientation of the drill guide 50. A locking screw 109 (FIGS. 14, 26) disposed in and along the approximately coronal axis of the pivot 64, enables a surgeon or technician to lock the parent bushing 53 into a selected buccal-lingual angular orientation (or pitch angle) 104 (FIG. 7).

At least one pilot or slip bushing 69 is provided for insertion into a bore 67 of the parent bushing 53. The slip bushing 69 has a cylindrical body 71 whose outer diameter is nearly equal to the internal diameter of the bore 67 of the parent bushing 53. It is also self-locking. The parent bushing 53 and yoke 52 are configured to form a seat 68 (FIG. 25). The slip bushing 69 includes an upper flange 70 adapted to fit within the seat 68 and, when seated, prevent the slip bushing 69 from rotating relative to the seat 68.

A first slip bushing 69 facilitates the drilling of a pilot hole into a jawbone. In some embodiments, additional slip bushings 69 are provided to facilitate the drilling of progressively larger holes. In other embodiments, the parent bushing 53 provides a sufficient guide for all of the progressively larger drill bits other than the pilot drill bit.

One of the many advantages provided by the dental device 20 is a procedure viewing window 100 (FIG. 11) formed in the platform 21 around the drill guide 50 and formed by the clearance between the drill guide 50 and the surgical entry point. The procedure viewing window 100 enables a dental surgeon to watch the drill bit as it contacts and drills into the edentulous area. The procedure viewing window 100 also facilitates flushing and evacuation of a patient's mouth within the procedure viewing window 100. The drill guide 50, which is configured to fit into gaps as small as 15 mm (or less) between adjacent teeth, is adapted for translational and rotational adjustment within the viewing window 100.

A form-fit bite cap (not shown), such as the bite cap depicted in Drawing Page 4 of the Provisional Application, is also provided that is configured to snap and be seated over the base of the platform 21. The form-fit bite cap is plastic and facilitates an X-ray or CT scan of the dental device 20 prior to dental osteotomy surgery.

There are mirror-image opposite layouts of the dental device 20. A dental device 20 according to a first layout is designed for operations on the right mandible and left maxilla. A dental device 20 according to a second layout is designed for operations on the left mandible and right maxilla.

In operation, a model 107 is created of a person's teeth. A stone drill or other marking device is used to mark an entry point on the model in an edentulous region corresponding to a desired location of the implant. A dental appliance 20 having at least one or more of the distinguishing features of the present invention, and of a size suitable for the dentition model, is selected. The primary and secondary receivers 26a and 26b of the tripod platform 21 of the dental appliance 20 are placed on opposite mesial and distal sides, respectively, of an edentulous region of the mouth. The cross-arch stabilizer 26c is positioned over a tooth portion (which may include multiple teeth) or gum ridge portion located on the side of the arch (left or right) opposite the side of the arch (right or left) where the secondary receiver is located. The cross-arch stabilizer 26c is then locked into this position using set screw (17).

The dental appliance 20 is removed and impression material (such as bite putty) is applied to the three bite registration receivers 26. The dental appliance 20 is then replaced on the model 107, conforming the impression material to the contours of the dentition or gum ridge under the three receivers 26.

After the impression material cures and hardens, a drill path alignment tool 101 is inserted through the drill guide 50 coupled to the dental appliance 20. The tip end 102 of the drill path alignment tool 101 is brought into contact with the marked entry point. While maintaining the tip end 102 in contact with the marked entry point, the drill path alignment tool 101 is moved to an angular orientation (both roll and pitch) that is coaxial with the desired angular orientation of the planned dental implant. The process of moving the drill path alignment tool translates and angularly adjusts the drill guide 50. The drill guide 50 is then mechanically locked (at least) into the desired position and angular orientation.

The bite cap is placed on the dental appliance 20, and the dental appliance 20 placed on the person's jaw. With the dental appliance and X-ray pin in place on the persons jaw, an image using cone beam X-ray tomography or other suitable imaging technology may be taken to verify that the entry point and orientation are suitable. If fine-tuning is merited, the dental appliance 20 is placed back on the model, the drill guide 50 unlocked, and—again using the drill path alignment tool 101 as a guide—calibrated adjustments (which may be computer controlled) made to the lateral position and angular orientation of the drill guide 50. The drill guide 50 is then again mechanically locked into the selected position and orientation. Furthermore, chemical adhesive or secondary locking screw is used to lock the carrier 49 to the platform 21.

The dental appliance 20 is now ready for use in guiding a surgical osteotomy. The dental appliance is placed back on the person's jaw. The dental appliance is placed on a person's jaw with a slip bushing 69 already inserted into the parent bushing 53. After a pilot hole is drilled, the slip bushing 69 is removed. Successively larger holes are drilled into the pilot hole using successively larger drill bits. The drill bits are simply inserted into the bore 67 of the parent bushing 53. Because the pilot hole has already been drilled at a precisely selected location and orientation, it is not expected that additional slip bushings 69 for the successively larger drill bits will be required. In one embodiment, however, one or more additional slip bushings 69 may be provided for one or more of the larger drill bits.

FIGS. 30-41 illustrate a third embodiment of a dental device 70 for guiding a drill bit, mounted on a model of a person's dentition 107. Like the dental devices 10 and 20 shown in previous embodiments, this dental device 70 comprises a platform or frame 71 adapted to be mounted on a person's jaw. Furthermore, the platform or frame 71 carries not just one adjustable drill guide 50, but a plurality of adjustable drill guides 50. There are also broad similarities between the carrier 49 depicted in the previous embodiments and the carriers 79 depicted in the present embodiment.

The platform 71 comprises three bite-registration receivers 76 (FIG. 34), the first (76a) of which is adapted to fit on a front area of the teeth or jaw, and a second and third (76b and 76c) of which are adapted to fit over rear tooth or ridge portions of the person's jaw. The receivers 76 are approximately coplanar with each other. Like the receivers 26 of the previous embodiment, each of these receivers 71 comprises a pad 29 with a plurality of putty receptacles or indentions 30 formed in the underside of the pad 29 for receiving an application of and retaining an impression material. The putty receptacles 30 are, in the depicted embodiment, formed with an undercut 31 to retain the impression material to the pad 29. After the impression material is conformed to a patient's dentition, the receptacles 30 retain the impression material to the receivers 71, forming anchors for the platform 71.

Here, the platform 71 comprises two mechanically deformable bars 73 linking the three receivers 71. The bars 73 are operable to be pulled apart into a wide configuration, or pressed together to a narrow configuration, to fit a variety of bite sizes. The recessed positions of the bars 73, deep within the mouth, provide ample procedure viewing windows 100 for the dental surgeon.

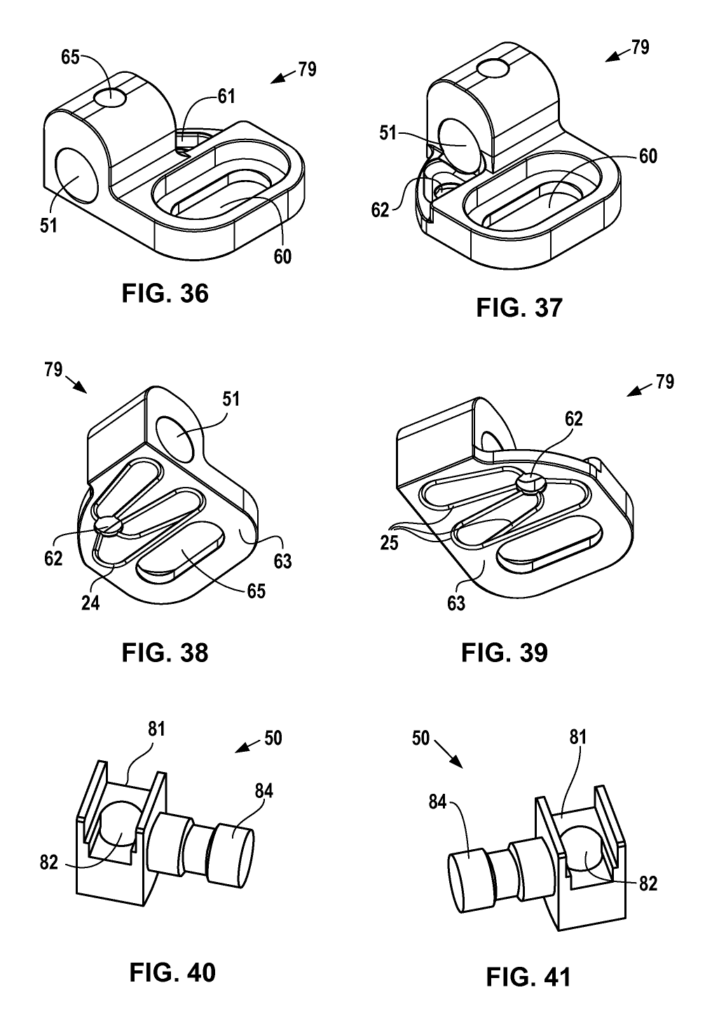

Turning to FIGS. 36-41, attention is now directed to aspects of the carrier 79 that couples the drill guide 50 to the platform 71. Each carrier 79 is pivotally and slidably joined via a pin-in-slot joint 60 to a corresponding receiver 76. The carrier 79 carries the drill guide 50 and translates it to a selected position between buccal and lingual limits 105 and between distal and mesial limits 106 across an approximately transverse plane. A slot 60 disposed in the carrier 79 and a substantially planar interface between the carrier 79 and the platform 71 facilitates selective translational movement of the carrier 79 via a pin-in-slot joint along a transverse plane. A mechanical lock 109, such as a locking screw and washer, enable the carrier 79 to be locked into a fixed position relative to the platform 21.

Substantially planar facing portions 63 and 23 of the carrier 79 and platform 71 also provide mating regions for applying adhesive to chemically lock the carrier 79 into a fixed position relative to the platform 21. An adhesive distribution system 24, such as a network of canals 25 positioned between a flat surface portion 23 of the platform 21 and a contacting surface portion 63 of the carrier 79, distribute the chemical adhesive between the carrier 79 and the platform 21 in order to lock the carrier 79 into a fixed position relative to the platform 21. A port 62 is also disposed in the carrier 79 to convey chemical adhesive from a top surface of the carrier 79 to a bottom surface of the carrier 79. A basin 61 is also disposed in the top surface of the carrier 79 for receiving and containing the adhesive and funneling some of the adhesive toward the port 62. The canal system 24 underneath the reservoir 61 and port 62 completes the distribution for an even displacement of adhesive between the carrier 79 and the main platform piece 22.

A drill guide 50 is disposed on the end of the carrier 79. In one embodiment, not shown, the drill guide 50 comprises a ball and socket joint. In another, consistent with that shown in the first and second embodiments, the drill guide 50 comprises a dual axle joint, such as a yoke 52 and parent bushing 53. In the embodiment shown in FIGS. 40 and 41, the drill guide 50 comprises a drill bushing housing 81, including a bore 82 for receiving a drill or pilot bushing 69 (FIG. 29), and a cylindrically-shaped pivot 84 for mounting in the carrier bore 51. In all of these embodiments, the carrier 79 and the drill guide 50 together form a multi-axis positioning assembly 48 for guiding the drill.

The drill guide 50 is pivotally coupled to the carrier 79 for buccal-lingual angular adjustment relative to the platform 71. A locking screw 109 disposed orthogonally of the carrier bore 51, through a vertical hole 65 disposed in the carrier 79, enables a surgeon or technician to lock the drill guide 50 into a selected buccal-lingual orientation (or roll angle) 103 (FIG. 7).

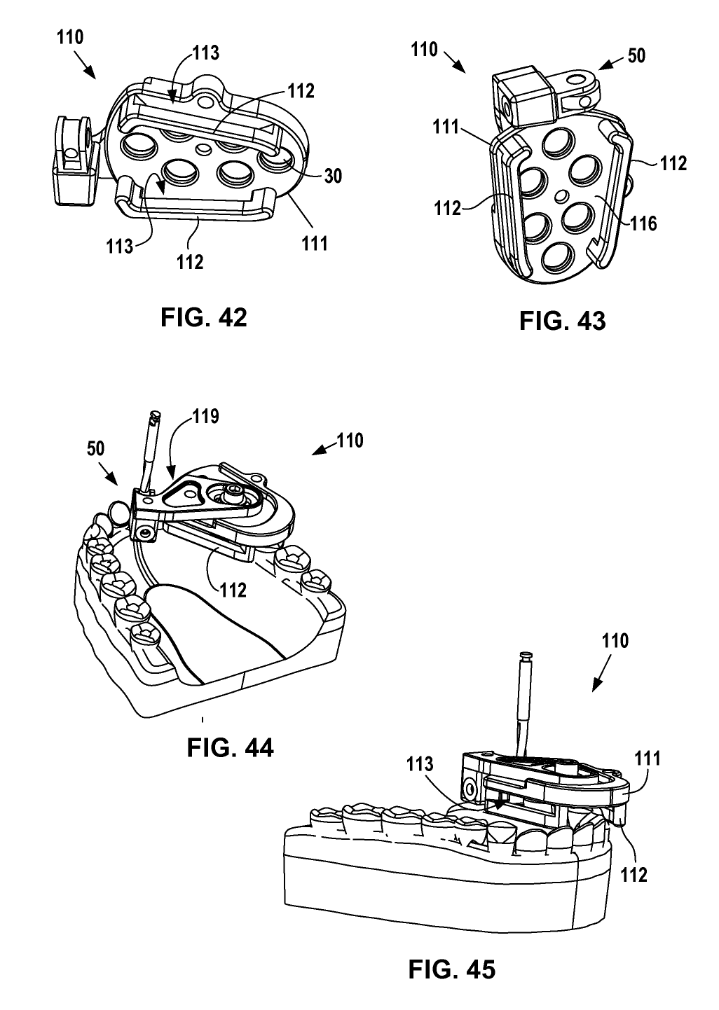

FIGS. 42-45 illustrate a dental device 110 like the dental device 10 of FIGS. 1-4 but with an improved drill guide platform or frame 111. The improved frame 111 forms a bite registration receiver 116 that includes downwardly-extending rails or braces 112 to bracket and brace the teeth and improve the stability of the dental device or appliance 110. FIGS. 42 and 43 are perspective bottom views of a left-sided dental device 110, and FIGS. 44 and 45 are perspective views of a right-sided dental device 110 mounted on a model 107 of a person's dentition. The braces 112 need not come into contact with the teeth or gum portion. Rather, the braces 112 provide lateral support to the dental putty that fits over the teeth. As seen best in FIGS. 42 and 45, a gap 113 between the brace 112 and the underside of the platform 111 or pad helps to secure the dental putty to the platform.

As with the earlier embodiments, an adjustable drill guide 50 is coupled to a carrier 119 to the frame 111. In the embodiments illustrated in FIGS. 42-45, the frame 111 includes bite registration receiver receptacles 30. In other embodiments, not shown, the receptacles 30 are excluded. The improvement illustrated in FIGS. 42-45 may be incorporated into the platforms of any of the preceding embodiments. One advantage of a frame 111 made with rails or braces 112 as shown in FIGS. 42-45, and particularly one that excludes the receptacles 30, is that it can be easily sterilized for reuse in subsequent dental surgeries, necessitating replacement only of the drill guide 50.

The present invention includes several independently meritorious inventive aspects and advantages. There are many different functional and structural aspects in the present disclosure that distinguish it from known prior art. However, it will be understood that the invention is not limited to embodiments that feature all of the disclosed functional and structural aspects. Rather, the invention encompasses a broad range of embodiments that feature one or more, but less than all of, the disclosed functional and structural aspects. For example, the invention encompasses both structures and methods. The methods are applicable to dental appliances other than the specific ones disclosed in the present specification. The invention also encompasses a wide variety of structural combinations, including dental appliances that couple an adjustable drill guide to a different kind of platform (other than an adjustable cross-arch platform), and dental appliances that use a combination of an adjustable cross-arch platform with a different kind of drill guide. The invention also broadly encompasses dental appliances that provide laterally translatable adjustable drill guides, dental appliances that provide a procedure viewing window for the surgical osteotomy operation, and dental appliances that provide locking receivers for impression material. In every case, the scope of any given claim will be set forth by the claim language itself.

When in the claims reference is made to a “tooth portion,” this may involve a portion of a single tooth or portions of multiple teeth.

Although the foregoing specific details describe various embodiments of the invention, persons reasonably skilled in the art will recognize that various changes may be made in the details of the apparatus of this invention without departing from the spirit and scope of the invention as defined in the appended claims.

I claim:

1. A dental device for guiding a drill bit, the dental device comprising:
a platform adapted to be mounted on a person's jaw; and
at least one drill guide coupled to the platform and adapted for translational and rotational adjustment with respect to the platform.

2. The dental device of claim 1, wherein the dental device provides at least a three-axis positioning assembly for the at least one drill guide.

3. The dental device of claim 1, further comprising an adjustable drill guide positioning assembly coupling the at least one drill guide to the platform.

4. The dental device of claim 1, wherein the at least one drill guide is operable to receive a drill path alignment tool and, while the drill path alignment tool's end is maintained in contact with a desired drill entry point, be adjusted by the drill path alignment tool into a desired angular orientation and lateral position with respect to the platform.

5. The dental device of claim 4, further comprising one or more mechanical locks to lock the at least one drill guide into the desired angular orientation and lateral position after the alignment tool has been used to set the desired angular orientation and lateral position.

6. The dental device of claim 5, wherein the dental device is operable to be placed on the person's jaw, imaged with respect to the person's jaw, removed from the person's jaw, and the mechanical locks are operable to be unlocked and re-locked to further adjust the at least one drill guide to a second, image-calibrated angular orientation and lateral position.

7. The dental device of claim 1, wherein the platform comprises a plurality of receivers, each receiver being adapted to fit over a tooth portion or ridge portion of the person's jaw.

8. The dental device of claim 7, wherein the plurality of receivers are adapted to be distributed across opposite sides of a dental midline of a person's jaw to stabilize the platform.

9. The dental device of claim 8, wherein first and second of the plurality of receivers are adapted to fit on distal and mesial sides of an edentulous area of the jaw, and a third of the plurality of receivers is a cross-arch receiver adapted to fit over a third tooth portion or ridge portion of the person's jaw.

10. The dental device of claim 9, wherein the third receiver is adjustably moveable relative to the first and second receivers.

11. The dental device of claim 10, wherein an angle between a line connecting the first and second receivers and a line connecting the first and third receivers is adjustable between narrow and wide angular limits to fit a variable range of mouth sizes and placements of the platform to a person's jaw.

12. The dental device of claim 7, wherein at least the first and second receivers are linked by a mechanically deformable bar, wherein deformation of the bar translates the drill guide along a coronal or apical direction relative to the person's jaw.

13. The dental device of claim 7, wherein the plurality of receivers are distributed on distal and mesial sides of multiple edentulous areas of the jaw.

14. The dental device of claim 13, wherein at least a first and second drill guide are coupled to the platform and adapted for and rotational adjustment with respect to the platform, the first drill guide is oriented over a first edentulous area of the jaw, and the second drill guide is oriented over a second edentulous area of the jaw.

15. The dental device of claim 13, wherein at least four drill guides are coupled to the platform and adapted for translational and rotational adjustment with respect to the platform, wherein first and second drill guides are coupled to the platform between a first pair of the plurality of receivers, and third and fourth drill guides are coupled to the platform between a second pair of the plurality of receivers.

16. The dental device of claim 7, wherein each receiver comprises a pad with one or more indentions formed in the pad for retaining impression material.

17. The dental device of claim 16, wherein the impression material is dental putty and the one or more indentions are putty receptacles.

18. The dental device of claim 18, wherein each putty receptacle is formed in the pad with an undercut to retain the putty to the pad.

19. The dental device of claim 1, wherein the drill guide comprises a drill bushing and an adjustable carrier, the carrier coupling the drill bushing to the platform.

20. The dental device of claim 1, wherein the platform comprises a plurality of bite registration receivers, each receiver being adapted to receive an application of impression material and, after the impression material is conformed to a patient's dentition, retain the impression material to the receiver.

21. The dental device of claim 1, wherein the platform comprises braces adapted to be placed on both sides of a tooth or gum portion of the jaw.

22. The dental device of claim 21, wherein the braces and underside of the platform are adapted to receive an application of impression material and, after the impression material is conformed to a patient's dentition, retain the impression material to the receiver.
